(19) United States
(12) Patent Application Publication
(10) Pub. No.: US 2019/0038797 A1
(43) Pub. Date: Feb. 7, 2019

(54) Sanitary Napkin

(71) Applicant: Paul Richard Van Kleef, Los Angeles, CA (US)

(72) Inventor: Paul Richard Van Kleef, Los Angeles, CA (US)

(21) Appl. No.: 15/666,502

(22) Filed: Aug. 1, 2017

(51) Int. Cl. A61L 15/44; A61L 15/40; A61L 15/18

(52) U.S. Cl. CPC A61L 15/44; A61L 15/40; A61L 15/18; A61L 2300/404; A61L 2300/10; A61L 2300/408; A61L 2300/30; A61L 2300/104; A61L 2300/406

(57) ABSTRACT

A sanitary napkin, towel, or pad having one or more of Manuka honey, Tourmaline powder, Jade powder, Amethyst powder, or silver applied to or sewn into a layer of the napkin.

### FIELD OF THE INVENTION

This invention relates to sanitary napkins. More particularly, this invention is directed to sanitary napkins containing products that confer health benefits.

### BACKGROUND OF THE INVENTION

Sanitary napkins, also referred to as sanitary pads or menstrual pads, are absorbent items worn by women to absorb blood or other fluid discharged from the vagina while menstruating, when recovering from vaginal surgery, during post-birth bleeding, or after an abortion. However, many women suffer from varying degrees of complications related to the vaginal discharge, including gynecological inflammation during menstruation due to changes in hormones that weaken the acidity of the discharge, loss of protein and other nutrients, and decreased immune system response.

### Manuka Honey.

Manuka honey, which is produced by bees that pollinate the native Manuka bush currently found in New Zealand, has natural antiviral and antibacterial qualities and may stimulate production of cells that can repair tissue damaged by infection. One antibacterial component in Manuka honey is methylglyoxal (MG). MG is stable and does not lose potency when exposed to heat or light. Other compounds in Manuka honey may also contribute to its medicinal effects.

The honey may be manufactured in a powdered form, with a consistency similar to powdered sugar. In powdered form, it is not sticky, and it does not have a sweet smell. The powder may be applied to fabric by, for example, spraying the powder onto fabric. To rate the potency of Manuka honey, honey producers employ a scale called the Unique Manuka Factor (“UMF”). A higher UMF rating indicates a more potent honey. A UMF rating of 10 or higher may be necessary to achieve certain physiological benefits.

### Far-Infrared and Negative Ions.

Ceramics emit infra-red radiation which depends on their temperature. Far-infrared (“FIR”) ceramic powders, nano-powders or nano-particles, which may emit wavelengths of approximately 15 micrometers, may be made from a variety of inorganic compounds. Small particles (nanoparticles and microparticles) of FIR-emitting ceramic material may be incorporated into fibers that are then woven into fabrics. They are non-toxic, tasteless, inorganic materials that can improve blood circulation, lower blood acidity, promote metabolism, and enhance immunity.

The mineral tourmaline, when milled into a fine powder, emits FIR and may be applied to the skin to improve blood flow. Jade powder and Amethyst powder also emit FIR. Heat from the human body may be transferred to these ceramic particles which maintain their temperature at sufficiently high levels, and then emit FIR back to the body. In this way, FIR-emitting material may prevent the loss of FIR that would otherwise be lost to the surrounding atmosphere.

Tourmaline particles and silver nanoparticles may also emit negative ions when, for example, pressure or heat is applied to the particles. Ions from silver may kill up to 650 types of bacteria. Together, far infrared radiation and negative ions nay soothe and detoxify the body.

### SUMMARY OF THE INVENTION

The present invention is directed to improved sanitary napkins. The improved sanitary napkins incorporate one more materials that provide health benefits, including Manuka honey, tourmaline powder, jade powder, amethyst powder, and silver.

It is an object of the present invention to relieve certain symptoms associated with vaginal discharge, including dysmenorrhea and premenstrual syndrome symptoms, using far-infrared and negative ion materials.

### BRIEF DESCRIPTION OF THE DRAWINGS

A further understanding of the invention can be obtained by reference to embodiments set forth in the illustrations of the accompanying drawings. Although the illustrated embodiments are merely exemplary of systems, methods, and apparatuses for carrying out the invention, both the organization and method of operation of the invention, in general, together with further objectives and advantages thereof, may be more easily understood by reference to the drawings and the following description. The drawings are not intended to limit the scope of this invention, which is set forth with particularity in the claims as appended hereto or as subsequently amended, but merely to clarify and exemplify the invention.

FIGS. 1A-1C depict illustrative sanitary napkins in accordance with the present invention.

FIG. 2 is a cross-section of an illustrative embodiment of a sanitary napkin of the present invention.

FIG. 3 is a cross-section of another illustrative embodiment of a sanitary napkin of the present invention.

### DETAILED DESCRIPTION OF THE INVENTION

The invention may be understood more readily by reference to the following detailed descriptions of preferred embodiments of the invention. However, techniques, systems, and operating structures in accordance with the invention may be embodied in a wide variety of forms and modes, some of which may be quite different from those in the disclosed embodiments. Consequently, the specific structural and functional details disclosed herein are merely representative, yet in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

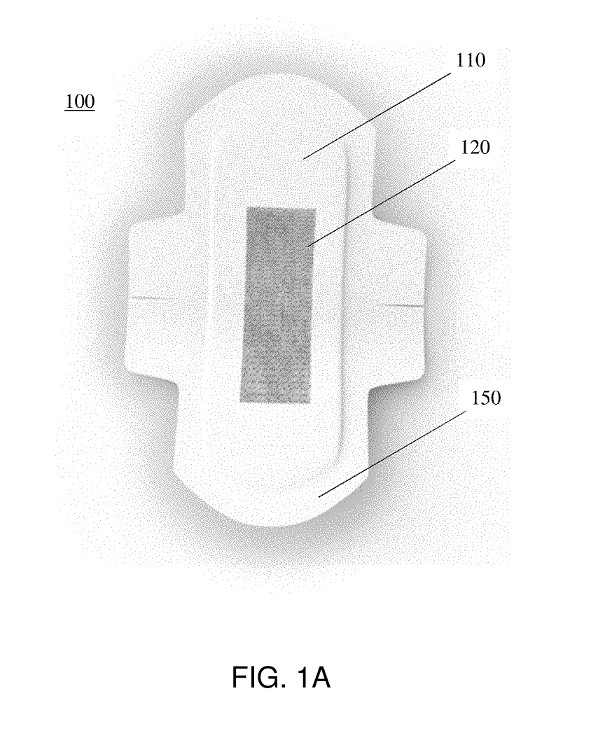

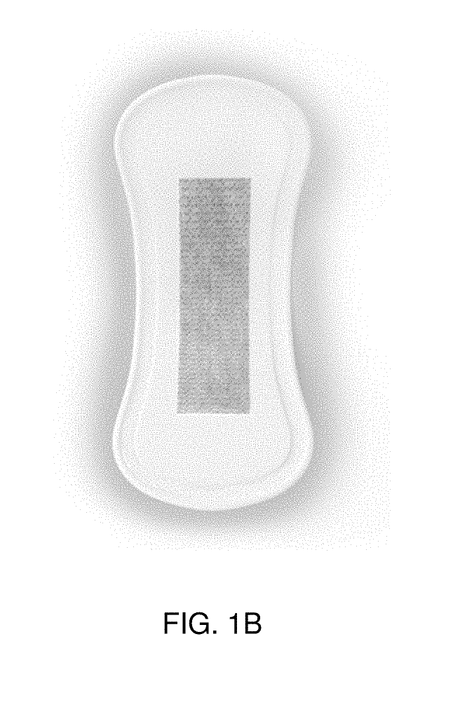

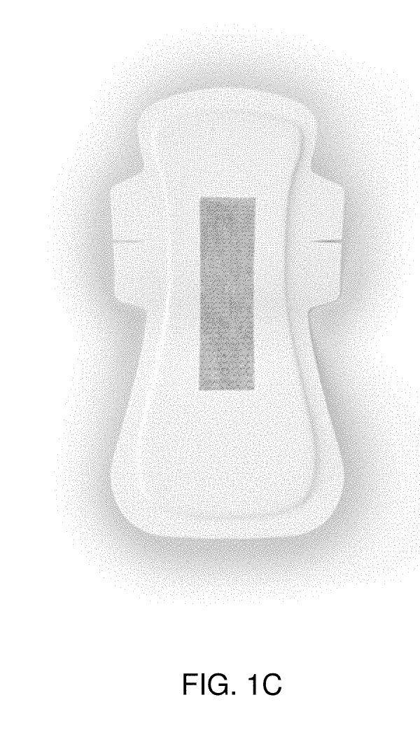

Referring to FIGS. 1A-1C, there are shown illustrative sanitary napkins in accordance with the present invention. Each sanitary napkin has a generally flat, multi-layer composition. Preferably the sanitary napkins are of a size and shape that are comfortable for wearing by women within an undergarment.

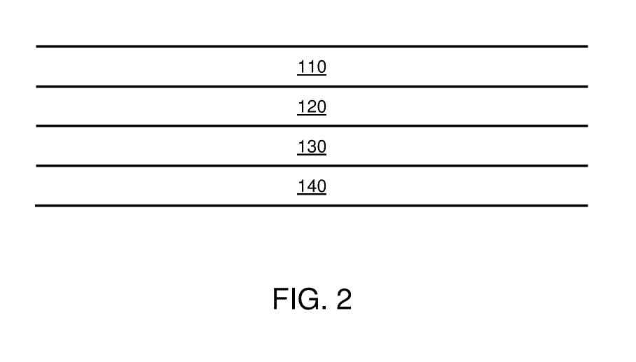

FIG. 2 depicts a cross-section of the layers of a sanitary napkin according to one embodiment of the present invention. Top layer (110), also referred to as the first layer, may be a liquid-pervious layer. Top layer (110) may have a mesh structure that allows liquid to pass through the holes of the mesh. In addition or in the alternative, top layer (110) may comprise a material such as cotton cloth that allows liquid to permeate through the material.

Second layer (120) may be positioned adjacent to top layer (110). Second layer (120) may be made from a fibrous, cloth material, such as cotton. One or more of Manuka honey, Tourmaline powder, Jade powder, or Amethyst powder may be applied to or sewn into second layer (120). For example, in one embodiment second layer (120) includes Manuka honey. In another example embodiment, second layer (120) includes Manuka honey and tourmaline powder. In another example embodiment, second layer (120) includes Jade powder. In another example embodiment, second layer (120) includes Jade powder and tourmaline powder. In another example embodiment, second layer (120) includes Amethyst powder. In another example embodiment, second layer (120) includes Amethyst powder and tourmaline powder. In each of the embodiments described above, second layer (120) may also include silver.

Second layer may be of the same shape and size as first layer (110), or may have a different shape and/or size. For example, second layer (120) shown in FIG. 1A has a rectangular shape that is smaller than first layer (110).

Third layer (130) may be positioned adjacent to second layer (120). Third layer (130) may comprise an absorbent material. Fourth layer (140), also referred to as the bottom layer, may be a liquid-impervious layer. Fourth layer (140) may be plastic or have a plastic coating. Fourth layer (140) may include FIR material. Fourth layer (140) may be positioned adjacent to third layer (130). Third layer (130) together with fourth layer (140) are intended to prevent any vaginal discharge from escaping the sanitary napkin.

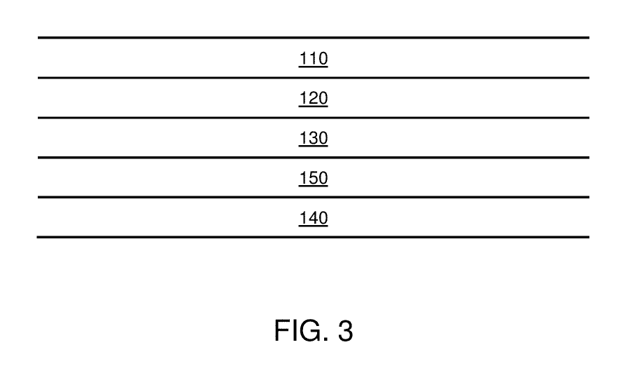

As shown in FIG. 3, fifth layer (150) may be positioned between third layer (130) and fourth layer (140). Fifth layer (150) may be a sponge material.

For each of the embodiments disclosed above, one or more of Manuka honey, Tourmaline powder, Jade powder, or Amethyst powder may be applied first layer (110) and/or bottom layer (140). The sanitary napkins according to the present invention may also have adhesives to facilitate positioning and wearing the sanitary napkin.

To realize the full health benefits of the sanitary napkin according to the present invention, the sanitary napkins may be worn up to six days prior to the start of menstruation, changing each sanitary napkin for a fresh sanitary napkin as necessary. Also, the sanitary napkins may be worn for at least six days after the start of menstruation, changing each sanitary napkin for a fresh sanitary napkin as necessary. Accordingly, the sanitary napkins according to the present invention may be worn for 12 consecutive days.

While the invention has been described with reference to the preferred embodiment and alternative embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention, therefore, shall be defined solely by the following claims. Further, it will be apparent to those of skill in the art that numerous changes may be made in such details without departing from the spirit and the principles of the invention. It should be appreciated that the invention is capable of being embodied in other forms without departing from its essential characteristics.

1-7. (canceled)

8. A sanitary napkin comprising:
a first, liquid-pervious layer, said first layer having an upper surface and a lower surface;
a second layer having an upper surface, a lower surface, and a first far infra-red emitting material, said first far infra-red material comprising Jade powder;
a third layer comprising an absorbent material, said third layer having an upper surface and a lower surface; and
a fourth, liquid-impervious layer, said fourth layer having an upper surface and a lower surface;
wherein the upper surface of the second layer is adjacent to the lower surface of the first layer, and the upper surface of the third layer is adjacent to the lower surface of the second layer.

9. The sanitary napkin of claim 8, further comprising a fifth layer wherein said fifth layer comprises a sponge material and said fifth layer has an upper surface and a lower surface.

10. The sanitary napkin of claim 9, wherein the upper surface of the fifth layer is adjacent to the lower surface of the third layer, and the upper surface of the fourth layer is adjacent to the lower surface of the fifth layer.

11. The sanitary napkin of claim 8, wherein the second layer further comprises a second far infra-red emitting material.

12. The sanitary napkin of claim 11, wherein the second far infra-red emitting material is tourmaline powder.

13. The sanitary napkin of claim 8, wherein the second layer further comprises silver.

14. The sanitary napkin of claim 8, wherein the fourth layer further comprises a second far infra-red emitting material.

15-20. (canceled)

16. The sanitary napkin of claim 12, wherein the second layer further comprises silver.

17. The sanitary napkin of claim 16, wherein the silver comprises silver nanoparticles.

18. The sanitary napkin of claim 13, wherein the silver comprises silver nanoparticles.

19. The sanitary napkin of claim 8, wherein the fourth layer further comprises a third far infra-red emitting material.

20. The sanitary napkin of claim 19, wherein the third far infra-red emitting material is tourmaline powder.

21. A sanitary napkin comprising:
a first, liquid-pervious layer, said first layer having an upper surface and a lower surface and a first far infra-red emitting material, said first far infra-red material comprising Jade powder;
a second layer having an upper surface, a lower surface, and a second far infra-red emitting material, said second far infra-red material comprising Jade powder;
a third layer comprising an absorbent material, said third layer having an upper surface and a lower surface; and
a fourth, liquid-impervious layer, said fourth_layer having an upper surface and a lower surface.

22. The sanitary napkin of claim 21, wherein the second layer further comprises a third far infra-red emitting material.

23. The sanitary napkin of claim 22, wherein the third far infra-red emitting material is tourmaline powder.

24. The sanitary napkin of claim 21, wherein the second layer further comprises silver.

25. The sanitary napkin of claim 24, wherein the silver comprises silver nanoparticles.

26. The sanitary napkin of claim 21, wherein the fourth layer further comprises a third far infra-red emitting material.

27. The sanitary napkin of claim 22, wherein the fourth layer further comprises a fourth far infra-red emitting material.
